(19) United States
(12) Patent Application Publication
(10) Pub. No.: US 2025/0161574 A1
(43) Pub. Date: May 22, 2025

(54) PEN-TYPE INJECTION DEVICES WITH MULTI-PIECE TELESCOPING PLUNGER ROD CONFIGURATIONS

(71) Applicant: Becton, Dickinson and Company, Franklin Lakes, NJ (US)

(72) Inventors: Alessandro PIZZOCHERO, Chelmsford, MA (US); Dana COTE, Kensington, NH (US); Mark WOOD, Sterling, MA (US); J. Richard GYORY, Sudbury, MA (US)

(73) Assignee: Becton, Dickinson and Company, Franklin Lakes, NJ (US)

(21) Appl. No.: 18/835,177

(22) PCT Filed: Jan. 31, 2023
(86) PCT No.: PCT/US2023/011934

§ 371 Date: Aug. 1, 2024

Related U.S. Application Data
(60) Provisional application No. 63/307,211, filed on Feb. 7, 2022.

(51) Int. Cl. A61M 5/315

(52) U.S. Cl. CPC A61M 5/31551; A61M 5/31593

(57) ABSTRACT

The disclosure provides driver components and methods of operating same that provide a reduced form factor for pen-type injection devices. In particular, various multi-piece telescoping plunger rod configurations are disclosed that can be implemented within pen-type injection devices to achieve such improvements. Driver components replace a single plunger rod having fixed length with telescoping pieces that are controlled to at least partially extend axially to dispense fluid from the reservoir or cartridge.

### CROSS-REFERENCE TO RELATED APPLICATIONS

This application is a 35 U.S.C. 371 National Stage Patent Application of PCT Patent Application No. PCT/US2023/011934, filed Jan. 31, 2023, which claims priority to U.S. Provisional Patent Application Ser. No. 63/307,211, filed Feb. 7, 2022, the entire contents of which are incorporated herein by reference.

### BACKGROUND

### Field

The disclosure relates generally to a multiple use pen-type injection device. More particularly, the present disclosure relates to driver components, systems and drive methods that facilitate reduced pen form factor, increased fluid chamber/reservoir/cartridge capacity, and improved dispensing accuracy in a multiple use pen-type injection device.

### Description of Related Art

Some medication (e.g., insulin) is self-administered. The typical diabetes patient will require injections of insulin several times during the course of a week or day. The required dose of insulin will vary from patient to patient, and for each patient may vary during the course of the day and from day to day. Usually, each diabetes patient will establish a regimen that is appropriate for his or her own medical condition and for his or her lifestyle. The regimen typically includes some combination of a slow or medium acting insulin and a faster acting insulin. Each of these regimens may require the diabetes patient to periodically self-administer insulin in public locations, such as places of employment or restaurants. The required manipulation of the standard hypodermic syringe and vial can be inconvenient and not sufficiently discreet for users to use in these public environments.

Medication delivery or injection pens have been developed to facilitate the self-administration of medication and are generally more discreet and convenient to use than syringes. An example of one such medication delivery pen is described in U.S. Pat. No. 5,279,585 (Balkwill), which includes a cartridge holder into which a cartridge of insulin or other medication may be received. The overall size of injection pens is impacted by the length of the cartridge and related components in the cartridge subassembly portion of a pen, and the length of the drive assembly for a plunger or piston rod in the pen body subassembly portion of a pen.

### SUMMARY

A need exists in the art for a pen-type injection device that has a smaller form factor and is therefore more convenient for a user to carry, while also providing accurate dose injecting. The above and other problems are overcome, and additional advantages are realized, by the illustrative embodiments described herein.

It is an aspect of illustrative embodiments to provide a medication injection pen, comprising: a chamber to hold fluid, the chamber comprising an outlet from which the fluid is expelled, and a stopper configured to advance within the chamber to push fluid from the chamber through the outlet to a patient; and a housing connected to the chamber and comprising a plunger rod subassembly configured to controllably move axially to move the stopper a selected amount in the chamber to expel a selected dose of the fluid. The plunger rod subassembly comprises at least two nested plunger rod members having a first plunger rod member slidably mounted within a second plunger rod member, and being controllably operated to move the first plunger rod member relative to the second plunger rod member to axially extend the plunger rod subassembly to push the stopper via a distal end of one of the nested plunger rod members.

In accordance with aspects of illustrative embodiments, the housing further comprises a dose set knob that rotates with respect to the housing during dose setting; and a driver selectively engaged with the dose set knob and threadably engaged with the plunger rod subassembly. The driver is rotatably fixed with respect to the housing during dose setting. The driver moves axially with the dose set knob during dose setting, rotates with the dose set knob during an injection to cause axial movement of at least one of the nested plunger rod members.

In accordance with aspects of illustrative embodiments, the chamber is a cartridge of fluid comprising the stopper and a subchamber holding the fluid. The length of the plunger rod subassembly is less than the length of the subchamber when in an initial nested position and at least as long as the length of the subchamber when extended from the nested position.

In accordance with aspects of illustrative embodiments, the first plunger rod member and the second plunger rod member are a plunger rod and a plunger rod inner member, respectively, that are arranged to be at least partially concentric with respect to each other such that the plunger rod at least partially surrounds at least a portion of the plunger rod inner member. The plunger rod and the plunger rod inner member are controllably operated to move telescopically with respect to each other.

In accordance with aspects of illustrative embodiments, the housing comprises a lead screw driver operable to be controllably rotated when the dose of fluid is to be delivered. The plunger rod inner member comprises inner threads that engage with outer threads on the lead screw driver to rotate when the lead screw driver rotates. The plunger rod inner member comprises outer threads that engage with inner threads in the plunger rod. The inner threads and outer threads of the plunger rod inner member, the inner threads in the plunger rod, and the outer threads on the lead screw driver have the same pitch.

In accordance with aspects of illustrative embodiments, the plunger rod inner member comprises distally located outer threads that engage with inner threads in the plunger rod, the outer threads being one of male threads and female threads and the inner threads being the other one of male threads and female threads.

In accordance with aspects of illustrative embodiments, the plunger rod is arranged within the housing to be prevented from rotating within the housing and relative to the plunger rod inner member. The housing further comprises a lead screw driver operable to be controllably rotated when the dose of fluid is to be delivered. The plunger rod comprises a counterbore at its proximal end that prevents a distal end of the plunger rod inner member from disengaging from the plunger rod. The plunger rod inner member comprises a cap at its distal end, inner threads that engage with outer threads on the lead screw driver to rotate when the lead screw driver rotates, and a coupling mechanism configured to provide temporary engagement between the plunger rod inner member and the lead screw driver until rotation of the plunger rod inner member causes the plunger rod to move axially to a point where the cap abuts the counterbore of the plunger rod and creates a force to disengage the plunger rod inner member from the lead screw driver and allow the plunger rod inner member to move axially.

In accordance with aspects of illustrative embodiments, the coupling mechanism on the plunger rod inner member comprises at least one detent on a proximal end thereof, the at least one detent configured to be frictionally engaged with a correspondingly-shaped and snap-fitted notch provided on a distal surface of the lead screw driver.

In accordance with aspects of illustrative embodiments, the housing comprises a brake tower axially and rotatably fixed thereto. The brake tower comprises a plurality of slots. The plunger rod comprises at least one tongue extending into one of the slots to engage the plunger rod with the brake tower. The plunger rod inner member comprises at least one tongue on its proximal end that enters the nearest one of the slots after the plunger rod inner member disengages from the lead screw driver to stop further rotation of the plunger rod and the plunger rod inner member and allow axial advancement of the plunger rod inner member as the leadscrew rotates.

In accordance with aspects of illustrative embodiments, the housing comprises a brake tower axially and rotatably fixed thereto. A proximal end of the plunger rod inner member and the brake tower have same handed threads that engage each other. The same handed threads being one of clockwise threads and counterclockwise threads. A distal end of the plunger rod inner member and the plunger rod have same handed threads that engage each other and are the other one of clockwise threads and counterclockwise threads such that the plunger rod and the plunger rod inner member move at the same time when a lead screw driver engaged with the brake tower and the plunger rod inner member rotates.

In accordance with aspects of illustrative embodiments, the threads on the plunger rod inner member, the plunger rod, and the brake tower have the same pitch.

In accordance with aspects of illustrative embodiments, the housing further comprises an anti-rotation sleeve disposed between the brake tower and the plunger rod. The anti-rotation sleeve comprises a slot. The brake tower and the plunger rod each comprise a stop member that is received in the slot.

In accordance with aspects of illustrative embodiments, an axial cross-section of the lead screw driver comprises at least a portion thereof that is shaped to be keyed into an internal portion of the plunger rod inner member.

Additional and/or other aspects and advantages of illustrative embodiments will be set forth in the description that follows, or will be apparent from the description, or may be learned by practice of the illustrative embodiments. The illustrative embodiments may comprise apparatuses having one or more of the above aspects, and/or one or more of the features and combinations thereof. The illustrative embodiments may comprise one or more of the features and/or combinations of the above aspects as recited, for example, in the attached claims

### BRIEF DESCRIPTION OF THE DRAWINGS

The above and/or other aspects and advantages of the illustrative embodiments will be more readily appreciated from the following detailed description, taken in conjunction with the accompanying drawings, of which:

FIG. 1A is a side view of an example injection pen, and FIG. 1B is a side view of a reduced length injection pen constructed in accordance with an example embodiment;

FIG. 2A is a side view of an example injection pen during dose setting, and FIGS. 2B and 2C are side views of a reduced length injection pen constructed in accordance with respective example embodiments;

FIG. 3A is a partial cross-section view of an example injection pen, and FIGS. 3B and 3C are partial cross-section views of a reduced length injection pen constructed in accordance with respective example embodiments;

FIGS. 4A and 4B are, respectively, exploded and cross-section views of an example injection pen;

FIG. 5 is a partial cross-section view of a pen body subassembly of an example injection pen;

FIG. 6 is a partial cross-section view of a pen body subassembly of an injection pen constructed in accordance with an example embodiment;

FIGS. 7A, 7B and 7C are partial cross-section views of an injection pen constructed in accordance with the example embodiment of FIG. 6 and in respective stages of axial movement;

FIG. 8 is a perspective view of a plunger rod and brake tower assembly constructed in accordance with the example embodiment of FIG. 6;

FIG. 9 is a partial side view of a plunger rod and lead screw assembly constructed in accordance with the example embodiment of FIG. 6;

FIG. 10 is a cross-section view of a plunger rod and brake tower assembly constructed in accordance with the example embodiment of FIG. 6;

FIG. 11 is a partial cross-section view of a pen body subassembly of an injection pen constructed in accordance with another example embodiment;

FIGS. 12A, 12B and 12C are partial cross-section views of an injection pen constructed in accordance with the example embodiment of FIG. 11 and in respective stages of axial movement;

FIG. 13 is a perspective view of a plunger rod and brake tower assembly constructed in accordance with the example embodiment of FIG. 11; and

FIGS. 14A and 14B are, respectively, a cross-section view and an exploded view of a plunger rod and lead screw assembly constructed in accordance with the example embodiment of FIG. 11.

Throughout the drawing figures, like reference numbers will be understood to refer to like elements, features and structures.

### DETAILED DESCRIPTION OF ILLUSTRATIVE EMBODIMENTS

Reference will now be made in detail to illustrative embodiments, which are depicted in the accompanying drawings. The embodiments described herein exemplify, but do not limit, the illustrative embodiments by referring to the drawings. The matters exemplified in this description are provided to assist in a comprehensive understanding of example embodiments of the disclosure. Accordingly, those of ordinary skill in the art will recognize that various changes and modifications of the embodiments described herein can be made without departing from the scope and spirit of the disclosure. Also, descriptions of well-known functions and constructions are omitted for clarity and conciseness.

Injection pens typically have a plunger rod similar to that in a syringe. The plunger rod is driven via a screw mechanism versus a linear force. Plunger length in existing pens is approximately equal to the length of the cartridge or barrel storing the fluid to be delivered in order to expel all of the fluid (e.g., medication) therefrom.

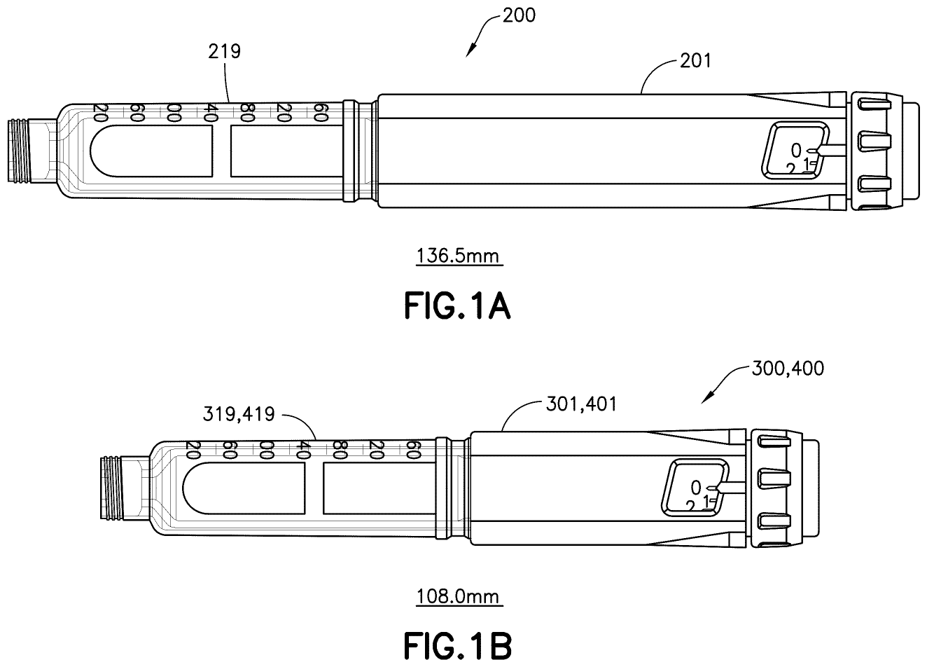

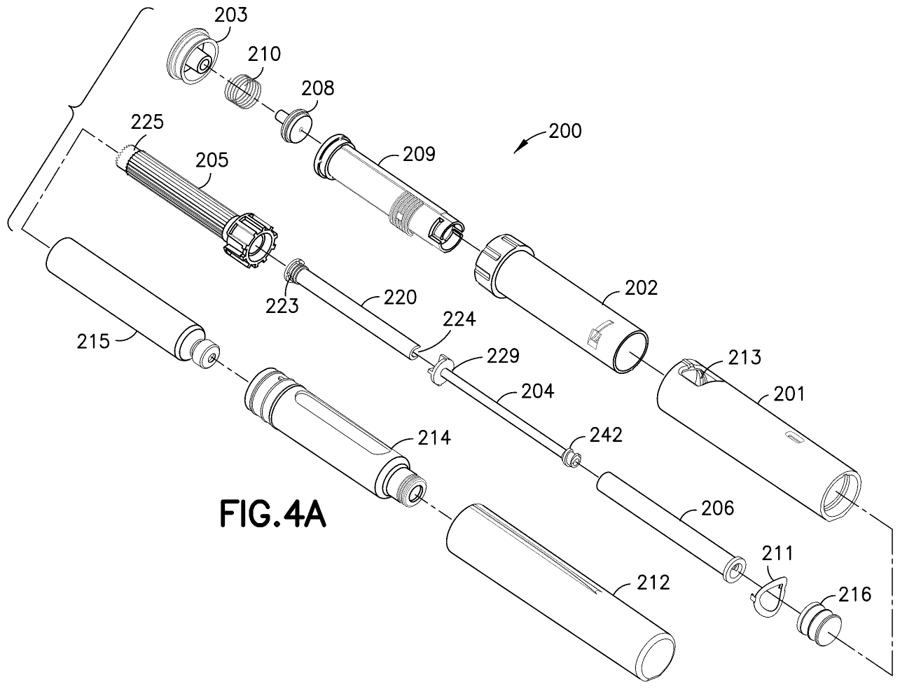

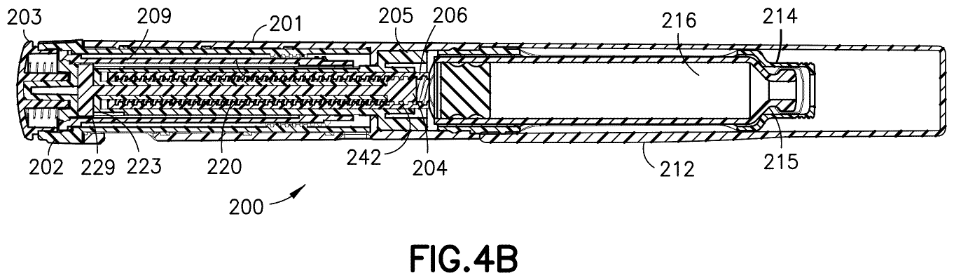

FIGS. 1A, 4AB and 4B depict, respectively, a side view, an exploded view, and a cross-section view of an example injection pen 200 that does not employ example embodiments of a telescoping plunger rod configuration to reduce pen form factor. The example injection pen 200 can be improved (e.g., the example injection pen 300 shown in FIG. 1B) in accordance with example embodiments described herein that employ respective telescoping plunger rod configurations to reduce its form factor, yet maintain or increase its volume of fluid to be delivered, and also realize increased dosing accuracy.

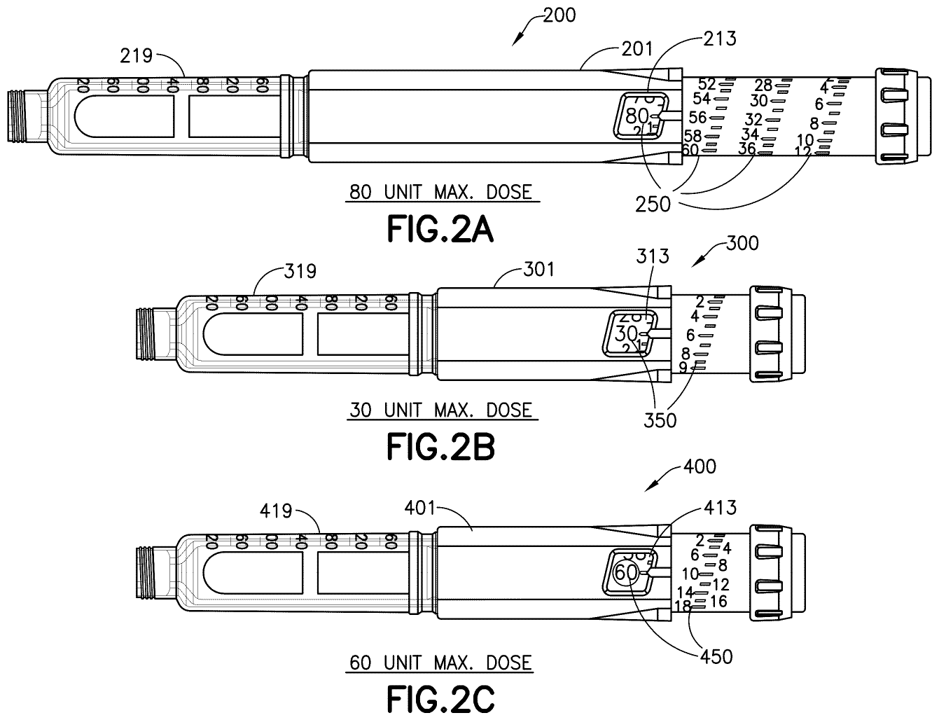

The example injection pen 200 shown in FIGS. 1A, 4A and 4B includes a pen upper body or housing 201, which houses a plurality of dose setting and injection components. The pen upper body 201 is connected to a cartridge housing 214, which houses a medication cartridge 215, as shown in FIGS. 4A and 4B. The injection pen 200 may also include a lower pen cap 212 to cover the cartridge 215 and cartridge housing 214 (e.g., an insulin cartridge subassembly 219) when the injection pen is not in use. As shown in FIG. 2A, the injection pen 200 includes a dose set knob 202 that includes a knob-like portion that is rotated by a user to set a desired dose visible in a window 213.

FIG. 4B depicts a cross-section of an example injection pen 200. Reference to the individual components may be better understood in view of the exploded assembly view shown in FIG. 1B. As shown, a push button 203 is provided at a proximal end of the pen upper body 201, closest to a user and farthest from a needle attached to the an insulin cartridge subassembly 219. The push button 203 can accommodate a spring 210 and is configured for a friction fit that maintains the push button 203 in a biased position on the dose set knob 202 under the force of the button spring 210, but allows the push button 203 to be pushed into the dose set knob 202 for injecting a set dose. The interior of the push button 203 accommodates a setback bearing insert 208 that rests on an internal surface at a proximal end of a setback member or driver 209. The push button 203 is designed to rotate freely on the setback bearing insert 208. A double clicker body 222 can be can be provided for audible feedback and is surrounded by the dose set knob 202. The double clicker can be provided with ratchet mechanisms to prevent rotation (e.g., clockwise) relative to the setback member 209 during dose setting via rotation (e.g., clockwise) of the dose set knob 202.

The setback member or driver 209 is a cylindrical member coaxial with and surrounded by the dose set knob 202. The setback member 209 is provided co-axially around a brake tower 205, which is axially and rotatably fixed to the pen upper body 201. The brake tower 205 co-axially surrounds a piston rod 206, as shown in FIG. 4A. A wave clip or spring 211, as shown in FIG. 4A, is provided between a distal end of the brake tower 205 and the cartridge 215 to bias the cartridge 215 in a distal direction to prevent any movement of the cartridge 215 during injection, and thus ensuring that an accurate dose is injected. As discussed further below, due to its threaded engagement with a lead screw 204, the piston rod 206 is moved into the cartridge 215 during injection to press on a stopper 216 provided inside the cartridge 215 to expel a dose of medication.

More specifically, a brake tower core 220 is surrounded by the brake tower 205 and is provided axially and rotationally fixed to the brake tower 205. The brake tower core 220 can be provided with a plurality of teeth provided on an enlarged surface 223 near the proximal end thereof. The plurality of teeth can extend axially toward a distal end, and are configured to engage corresponding teeth 225 provided at a proximal end of the brake tower 205. The corresponding tooth engagement prevents relative rotation between the brake tower core 220 and the brake tower 205. The brake tower 205 is both axially and rotationally fixed to the pen upper body 201 in the same manner described above. As shown, the brake tower core 220 is a substantially cylindrical element with an open side 224 extending along an axial length of the brake tower core 220. The open side 224 can include, for example, approximately one-fifth to one-quarter of the circumference of a cross section of the brake tower core 220. The open side 224 therefore forms two longitudinally extending edges at each end of the open side 224.

The brake tower core 220 functions to prevent rotation of the piston rod 206 relative to the brake tower 205 and thus the pen upper body 201. The brake tower core 220 is surrounded by a hollow piston rod 206. The hollow piston rod 206 includes a plurality of thread segments provided along substantially the entire length of the hollow piston rod 206. Each of the thread segments has a length substantially the same as the portion of the circumference of the open side 224 of the brake tower core 220. The thread segments extend inwardly into the inner cavity of the hollow piston rod 206. The piston rod 206 is positioned with respect to the brake tower core 220 such that its thread segments align with and protrude into the open surface 224 of the brake tower core. In this position, the pair of longitudinally extending edges formed by open side 224 abut the respective edges of the protruding thread segments, such that the piston rod 206 is prevented from rotating relative to the brake tower core 220.

Similar to the above exemplary embodiments, a lead screw 204 is provided in the interior of the hollow piston rod 206. A threaded portion 242 is provided at the distal end of the lead screw 204. Threaded portion 242 is configured to engage the thread segments provided on the interior of the piston rod 206. The lead screw 204 is rotationally fixed to a setback member 209 such that rotation of the setback member 209 during an injection is transferred to the lead screw 204. Axial movement of the lead screw 204 relative to the brake tower core 220 is prevented in the proximal direction by the lead screw 204 threads 242 being larger than the diameter of the opening at a distal end of the brake tower core 220. Axial movement of the lead screw 204 relative to the brake tower core 220 is prevented in the distal direction by a flange 229 of the lead screw 204 engaging the enlarged portion 223 of the brake tower core 220. As such, due to the thread engagement between the threaded portion 242 of the lead screw 204 and thread segments on the hollow piston rod 206, relative rotation of the lead screw 204 with respect to the piston rod 206 (which is rotationally fixed to the brake tower 205) drives the piston rod 206 axially in the distal direction inside the cartridge 215 to expel medication contained therein.

Reference is now made to FIG. 1B, which depicts an improved, shortened injection pen 300 constructed in accordance with an example embodiment wherein the piston rod 206, also referred to as a plunger rod, is converted from singular plunger rod 206 to a two-piece telescoping configuration. When comparing FIGS. 1A and 1B to each other, it is clear that the dimensions of the insulin cartridge subassemblies 219 and 319 are the same in terms of circumference and length; however, the pen upper bodies 201 and 301 have the same circumference but their lengths are different with the length of the upper body 301 of the pen being advantageously shortened for reduced form factor, among other benefits. For example, the pen 200 in FIG. 1A and the pen 300 in FIG. 1B are both shown with their respective lower pen cap 212 and 312 removed and with no needle assembly installed at the distal end of their respective insulin cartridge subassembly 219 and 319. Pen 200 in FIG. 1A, however, is 136.5 millimeters (mm) long while pen 300 in FIG. 1B is only 108.0 mm long. It is to be understood that the housing 301 and its piston driver assembly components can have different lengths than that shown in FIG. 1B.

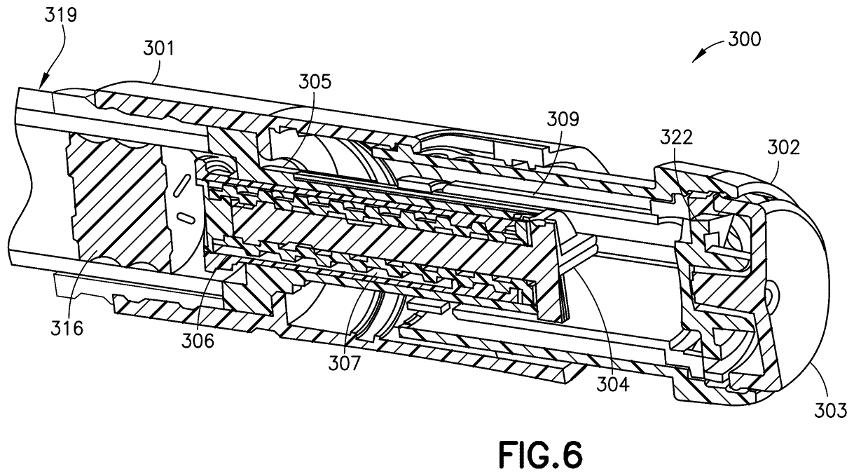

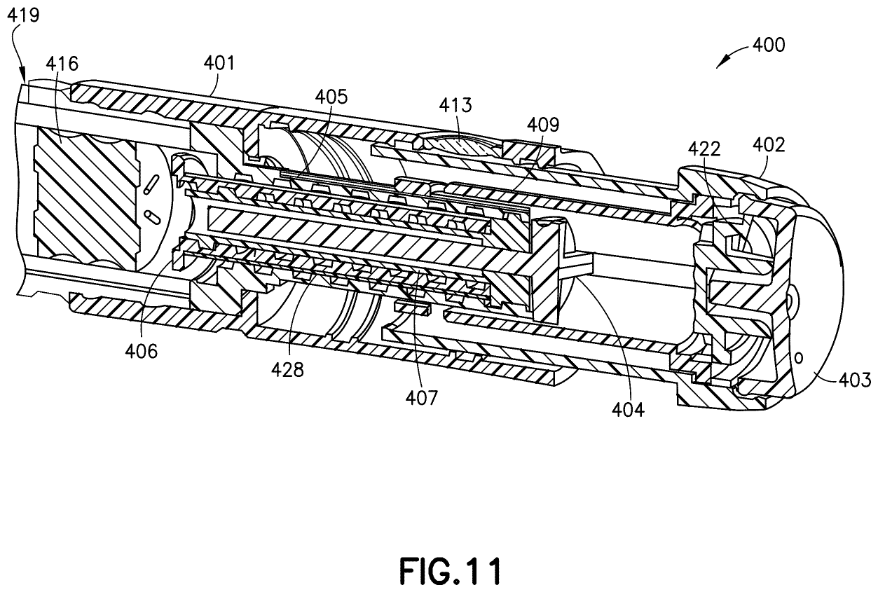

As described below in connection FIGS. 6-10, the pen 300 can comprise a two-piece plunger rod with double threaded telescoping screw-train configuration in accordance with an example embodiment (hereinafter referred to as the “double thread” telescoping screw-train embodiment or pen 300) to achieve the same axial movement of the stopper 316 as the stopper 216 moved by the singular piston rod 206 in pen 200, but without requiring as much length in the housing 301 to accommodate piston driver assembly components. As described below in connection FIGS. 11-14, the pen 300 can alternatively be implemented as another two-piece plunger rod with double-acting telescoping screw-train configuration in accordance with another example embodiment (hereinafter referred to as the “double-acting” telescoping screw-train embodiment or pen 400) to achieve the same axial movement of the stopper 316 as the stopper 216 moved by the singular piston rod 206 in pen 200, but without requiring as much length in the housing 301 to accommodate piston driver assembly components.

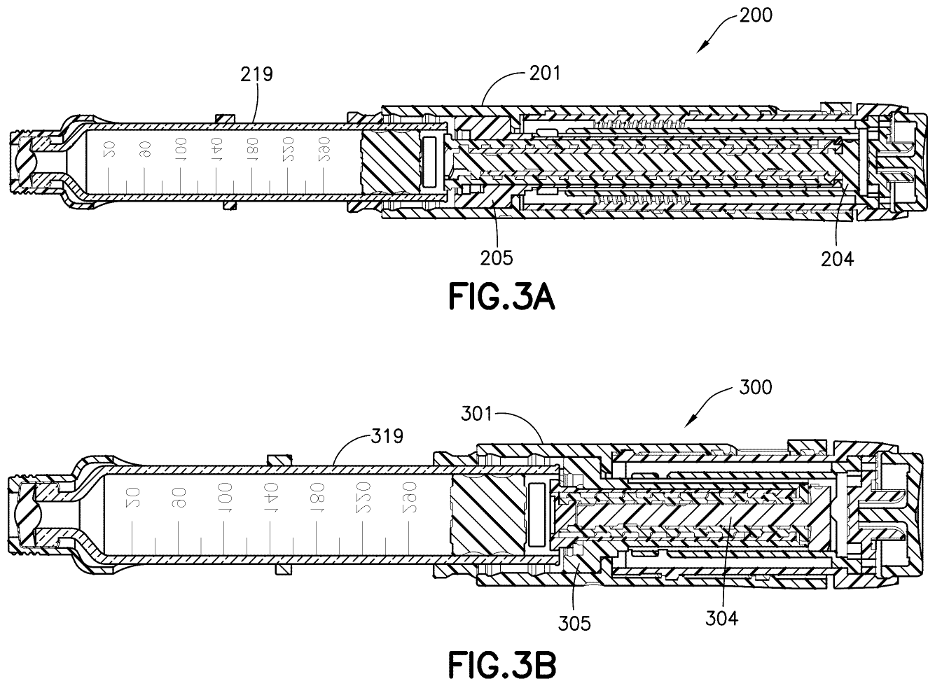

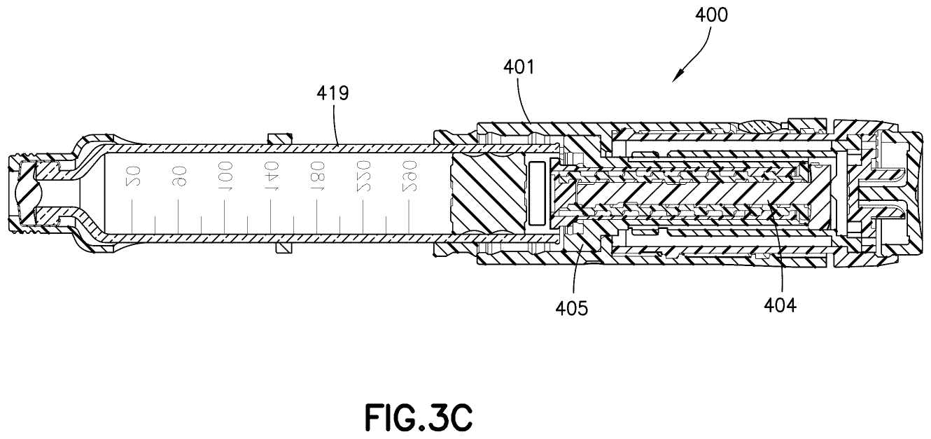

Additional comparisons with respect to the reduced form factors of the respective double thread and double-acting telescoping screw-train embodiments are depicted in FIGS. 2A, 2B and 2C and in FIGS. 3A, 3B and 3C. FIGS. 2A, 2B and 2C illustrate, respectively, exterior side views of a standard length injection pen 200, an advantageously reduced form factor double thread injection pen 300, and an advantageously reduced form factor telescrew injection pen 400, in a dose setting mode. FIGS. 3A, 3B and 3C illustrate, respectively, cross-section side views of a standard length injection pen 200, an advantageously reduced form factor double thread injection pen 300, and an advantageously reduced form factor double-acting injection pen 400, in a pre-dose setting mode.

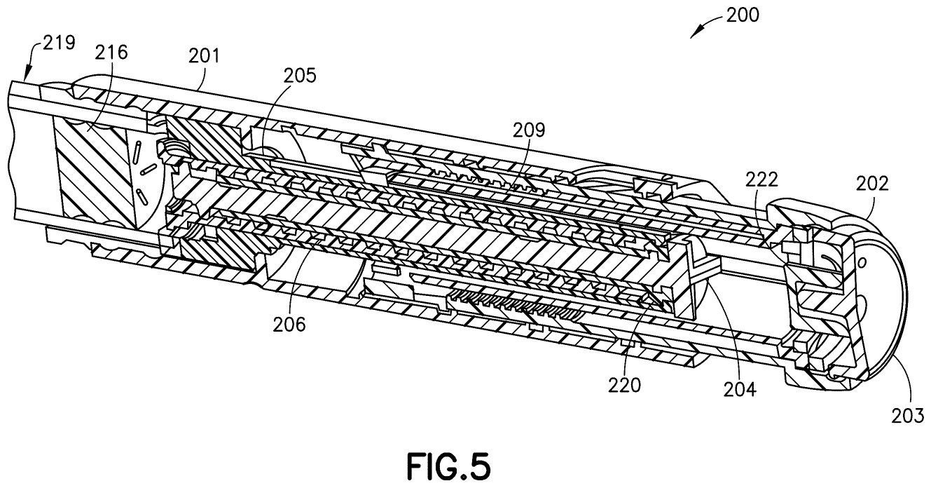

Before describing the respective double thread and double-acting embodiments below, dose setting operation, and dosing (i.e., injection) operation, of a standard length injection pen 200 shall be described with reference to FIG. 5. To set a dose, a user rotates the dose set knob 202 clockwise (CW) to the desired dose amount. The setback member 209 remains in its current angular position, because its ratchet mechanism with the brake tower 205 only allows counter-clockwise (CCW) rotation. If the user rotates the dose set knob 202 CCW, the setback member 209 remains in the same angular position because there is no axial force locking the male detents in the setback member 209 into corresponding female detents the dose set knob 202. The double clicker 222 can ratchet mechanisms prevent CW rotation relative to the setback member 209, as well as create audible feedback (e.g., clicking sounds) during and corresponding rotation of the dose set knob 202.

With continued reference to FIG. 5, to perform dosing, a user pushes on the button 203. The dose set knob 202 starts to rotate CCW as its male thread screws into a corresponding female thread in the body 201. The setback member 209 also rotates CCW because the force that was created by the user pressing button 203 locks the male detents of the setback member 209 into the female detents on the dose set knob 202. The lead screw 204 also turns CCW because its head has two protuberances that are engaged with longitudinal slot in the setback member 209. The lead screw 204, when rotating, threads into the piston rod 206 to advance it and ultimately the stopper 216 a selected axial distance to administer the desired dose of insulin. The tower core 220 is keyed into the brake tower 205 and the piston rod 206 so that the piston rod 205 cannot spin, ensuring relative motion between the lead screw 204 and the piston rod 206.

In accordance with the example double thread embodiment illustrated in FIGS. 6-10, an improved injection pen 300 realizes a reduced form factor by using a two-piece plunger rod with double thread telescoping screw-train configuration. With reference to FIG. 6, the pen 300 comprises a body 301 with dose setting and dosing components, and a fluid cartridge subassembly 319. The components 302, 303, 322, 316 and 319 can be similar to respective components 202, 203, 222, 216 and 219 described above. A modified plunger rod comprises two pieces, that is, a plunger rod 306 and a plunger rod inner member 307, that are each shorter in length than the piston rod 206. Correspondingly, the body 301, lead screw 304 and brake tower 305 in the pen 300 are also shorter in length than corresponding parts 201, 204 and 205 in the pen 200. The plunger rod 306 and the plunger rod inner member 307, however, are telescopically arranged and threaded to extend to an overall length that is commensurate with the piston rod 306 to move stopper 316 the same overall distance in the cartridge of the cartridge subassembly 319 as the piston rod 206 moves the stopper 216 in a cartridge subassembly 219 having the same volume length dimensions as cartridge subassembly 319.

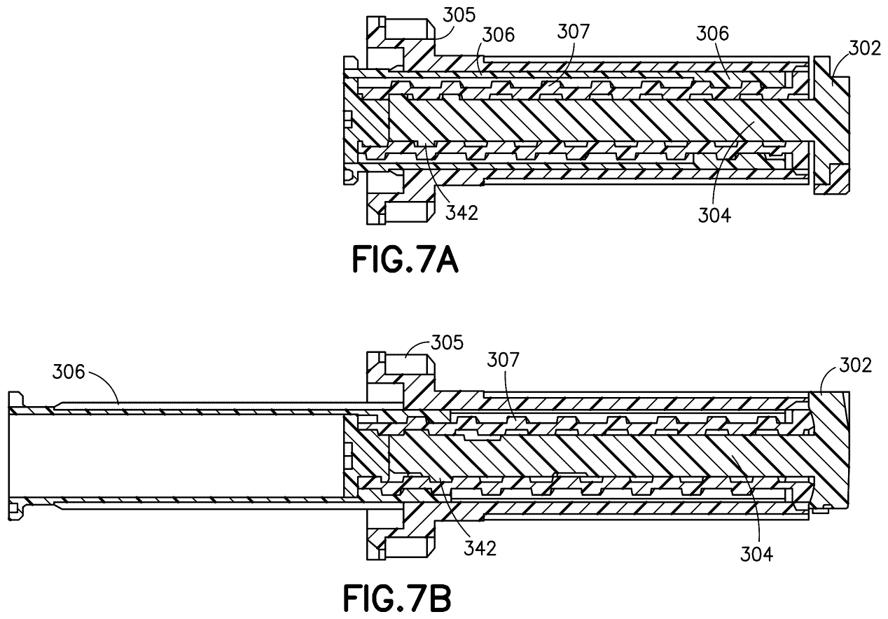

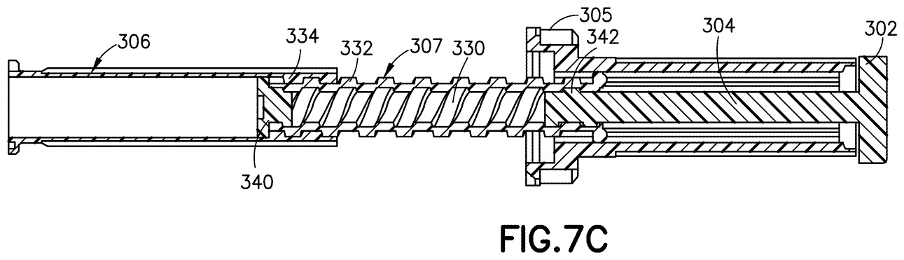

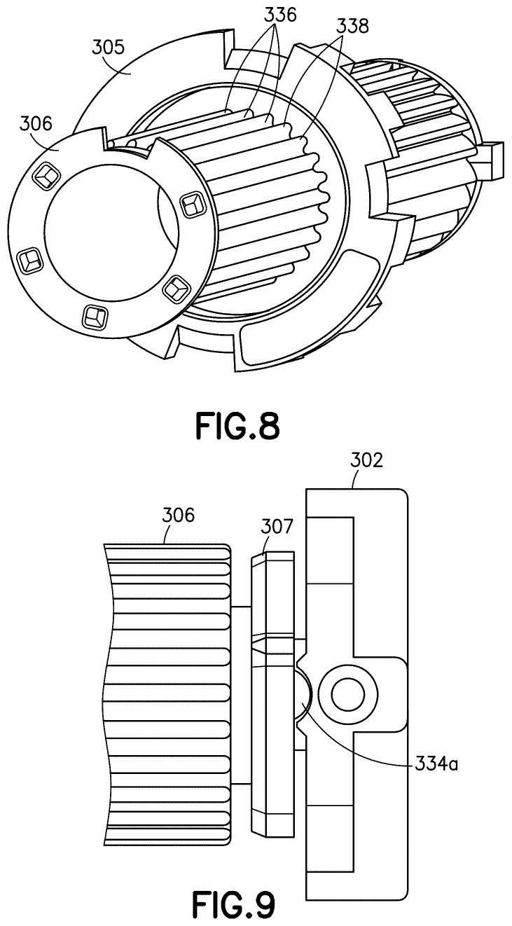

For example, as shown in FIGS. 6-10, the plunger rod inner member 307 can be provided with inner threads 330 that engage threads 342 exterior of the lead screw 304. Outer male threads 332 engage with female threads 334 provided on the plunger rod 306, as shown in FIGS. 7A-7C. Comparing pen 300 to pen 200, the threads 334 are repositioned compared to threads in piston rod 206. Further, the tower core 220 is obviated. Instead, the example embodiment employs a set of tongues 336 and grooves 338 on the plunger rod 306 and the brake tower 305 to stop the plunger rod 306 from rotating, as illustrated in FIG. 8. A cap 340 is provided to the end of the plunger rod inner member 307 to facilitate threading of the pen body subassembly in production and to provide a vertical face for stopping advancement of the plunger rod inner member before it unscrews itself from the plunger rod 306. This telescoping configuration allows the plunger rod subassembly 306,307 to nearly double in length (FIG. 7C) compared to its initial nested position shown in FIG. 7A. The plunger rod 306 moves in the same increments in pen 300 as the piston rod 206 in the pen 200 because the plunger rod threads 334 move one at a time and have the same pitch as threads in the piston rod 206.

With continued reference to FIGS. 6-10, after setting a desired dose with the dose set knob 302, a user pushes on the button 303 to commence dosing. The dose set knob 302 begins to rotate CCW, as its male thread screws into the body 301's female thread. The setback member 309 also rotates CCW because the force applied to the button 303 locked the setback member 309's male detents into the dose set knob 302's female detents. The lead screw 304 also turns CCW because its head has two protuberances that are engaged with the longitudinal slots in the setback member 309. The rotating lead screw 304 threads into the plunger rod inner member 307 to advance it, the plunger rod 306 and ultimately the stopper 316 to administer the desired dose of fluid from the cartridge (e.g., insulin).

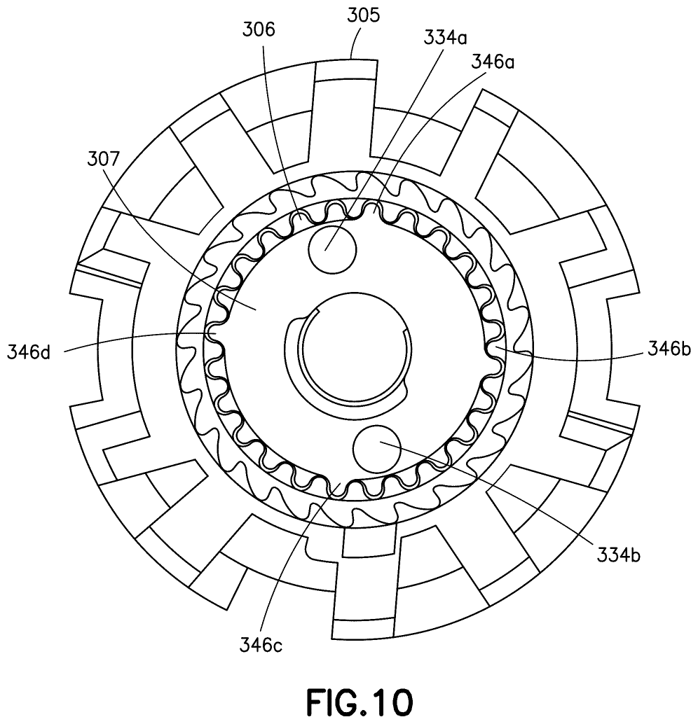

The plunger rod inner member 307 is configured such that the plunger rod 306 translates first, then the plunger rod inner member 307 after bottoming out in the counterbore. The lead screw 304 is temporarily coupled to the plunger rod inner member 307 via dual detents 344a, 334b, as shown in FIG. 9 to ensure the desired timing. Once the underside of the cap 340 on the plunger rod inner member 307 bottoms out on the end of the plunger rod 306's counterbore (FIG. 7B), the threading forces will create enough rotational force to release the dual detents 344a, 334b. Four tongues 346a, 346b, 346c and 346d on the plunger rod inner member 307 will enter the nearest set of brake tower grooves 338, as shown in FIG. 10, thereby stopping rotation of both plunger rod pieces 306,307 and forcing advancement as the lead screw 304 turns.

In accordance with another example embodiment, a double-acting telescoping screw-train configuration will now be described with reference to a pen 400 as shown in FIGS. 11-14. In accordance with this embodiment, a pen 400 has a modified plunger rod to allow for an advantageous reduced form factor pen. For example, a plunger rod such as the piston rod 206 in pen 200 has been modified in the pen 400 as a two-piece telescoping screw-train comprising a plunger rod 406 and a plunger rod inner member 407, that are each shorter in length than the piston rod 206. Correspondingly, the body 401, lead screw 404 and brake tower 405 in the pen 300 are also shorter in length than corresponding parts 201, 204 and 205 in the pen 200. The plunger rod 406 and the plunger rod inner member 407, however, are telescopically arranged and threaded to extend to an overall length that is commensurate with the piston rod 206 to move stopper 416 the same overall distance in the cartridge of the cartridge subassembly 419 as the piston rod 206 moves the stopper 216 in a cartridge subassembly 219 having the same volume length dimensions as cartridge subassembly 419.

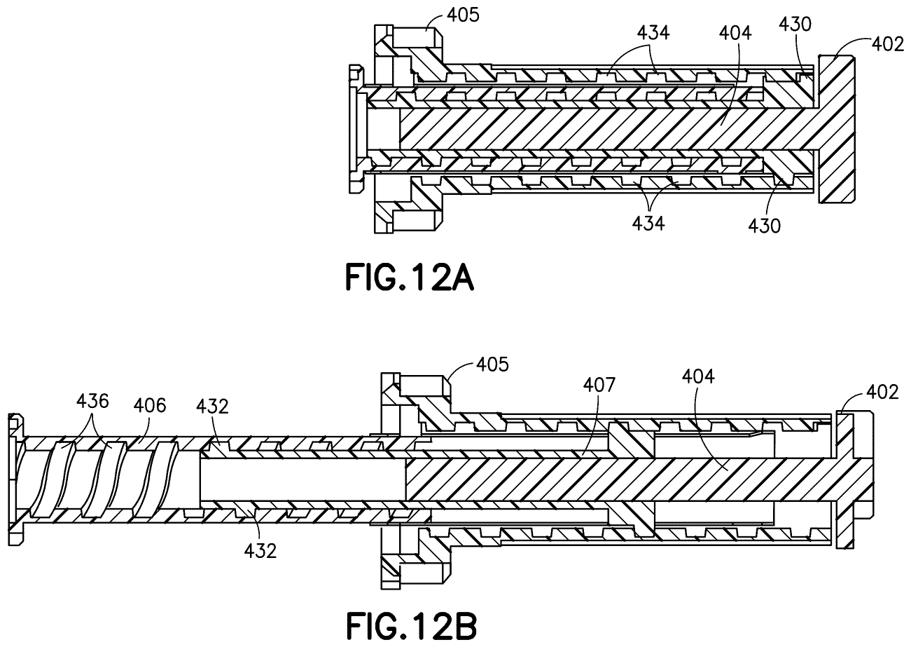

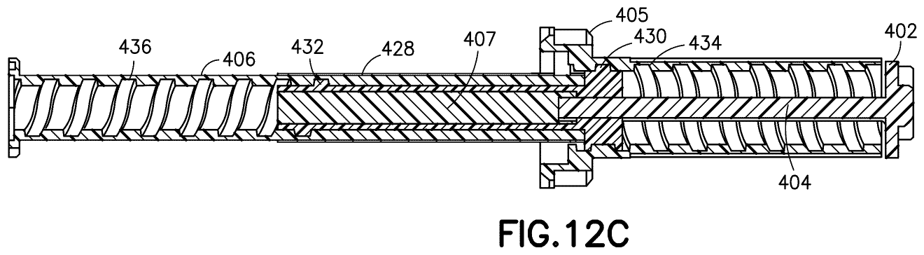

For example, as shown in FIGS. 12A-12C, the plunger rod inner member 407 can be provided with proximal male CCW threads 430 that interface with female CCW threads 434 in the brake tower 405 and distal male CW threads 432 that interface with the female CW threads 436 in the plunger rod 406. This telescoping arrangement allows the plunger rod subassembly 406, 407 to nearly double in length when extended (FIG. 12C) compared to its initial nested position (FIG. 12A). While threads in the plunger rod 406 and the plunger rod inner member 407 have the same pitch as threads in the piston rod 206, for example, both the plunger rod 406 and the plunger rod inner member 407 move at the same time because they have opposite thread directions, causing the plunger rod subassembly 406, 407 to move twice the distance per lead screw revolution 404, as compared to piston rod 206 movement by the lead screw 204 in the pen 200.

In accordance with another aspect of the example embodiment of a pen 400 with a double-acting telescoping screw-train configuration as shown in FIGS. 11-14, the text or indicia on the dose set knob 402 is modified to reflect the doubling in motion of the plunger rod subassembly 406, 407. FIGS. 2A, 2B and 2C illustrate, respectively, windows 213, 313, 413 with indicia 250, 350, 450 (e.g., indicating 80 unit maximum dose, 30 unit maximum dose, and 60 unit maximum dose). For example, as illustrated at 450 in a window 413 of the pen 400 in FIG. 2C, each line now represents 2 units of insulin compared, for example, to 1 unit as indicated on the pen 200 in FIG. 2A. Thus, text or other indicia, in turn, is added to every line in FIG. 2C, versus every other line as shown in FIGS. 2A and 2B. The pen 400 can be implemented to decrease the font size of the indicia 450 to avoid having disparate indicia overlap. Also, a magnifying lens can be used at the window 413 to aid the readability of the dose units to the end user. Similarly, a magnifying lens can be used at the window 313 of the pen 300 in FIG. 2B to aid the readability of the indicia 350 for dose units to the end user.

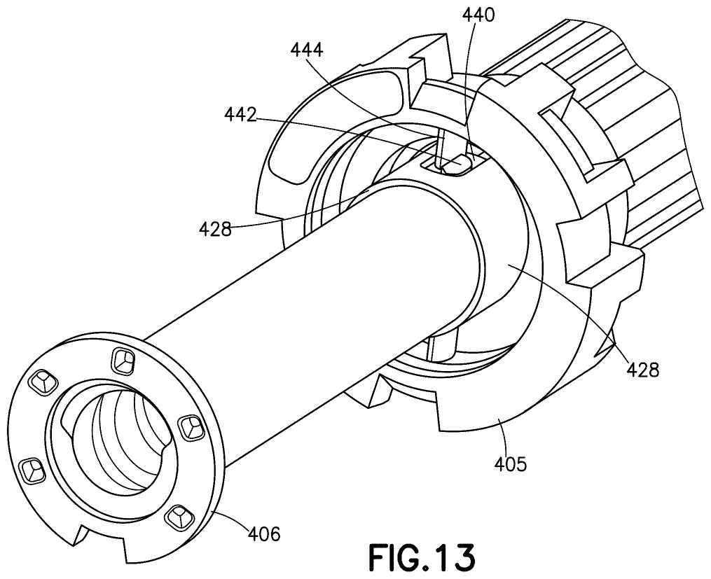

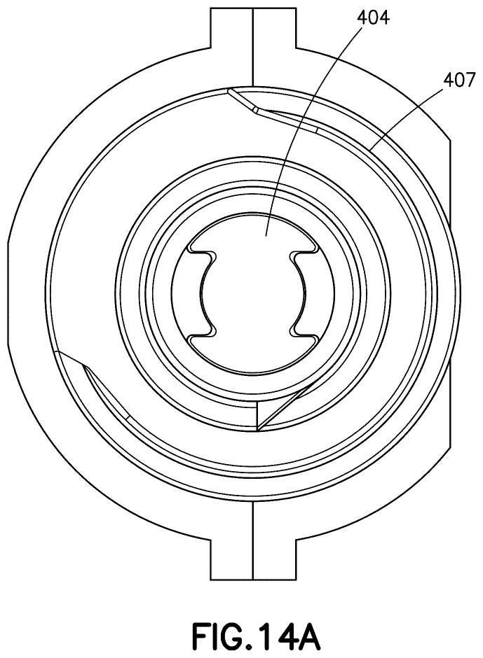

With continued reference to FIGS. 11-14, a stainless steel anti-rotation sleeve 428 has been provided in the pen 400. The anti-rotation sleeve 238 comprises a set of slots 440. Two sets of stops 442 and 444 provided, respectively, on the plunger rod 406 and the brake tower ride inside the set of slots 440 of the anti-rotation sleeve 428 to prohibit plunger rod 406 rotation, as illustrated in FIG. 13.

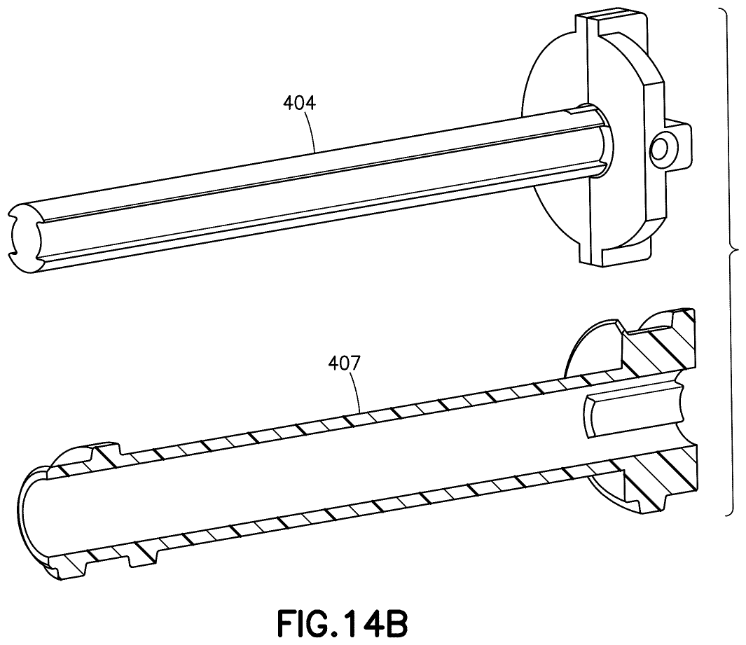

With continued reference to FIGS. 11-14, after setting a desired dose with the dose set knob 402, a user pushes on the button 403 to commence dosing. The dose set knob 402 begins to rotate CCW, as its male thread screws into the body 401's female thread. The setback member 409 also rotates CCW because the force applied to the button 403 locked the setback member 309's male detents into the dose set knob 302's female detents. The lead screw 404 also turns CCW because its head has two protuberances that are engaged with the longitudinal slots in the setback member 409. The lead screw 404 is slidably keyed into the plunger rod inner member 407 causing them to rotate together, as illustrated in FIGS. 14A and 14B. As the plunger rod inner member 407 rotates, it advances distally because of its threaded relationship with the brake tower 405. Simultaneously, the plunger rod 406 advances because of its threaded relationship with the distal male thread 432 on the plunger rod inner member 407. Plunger rod 406 advancement on the stopper 416 administers the desired dose of fluid from the cartridge (e.g., insulin).

In accordance with example embodiments of the present disclosure, an injection pen (e.g., pens 300 and 400) can deliver a full 300 units of insulin from a cartridge 214, for example, using a pen body (e.g., pen bodies 301 and 401) that is 40% shorter than the body 201 of a pen 200 that does not employ the technical solution of a two-piece, telescoping screw-train provided by the example embodiments to advance a stopper (e.g., stopper 216). Thus, significant length reduction of about 20% of the injection pen 300, 400 and corresponding space savings is realized by the example embodiments to increase convenience to the user.

Due to space limitations within many injection pens, the example embodiments have been illustrated with dual screw-train arrangements. It is to be understood, however, the example embodiments of the present disclosure can be implemented using a triple screw-train arrangement. In addition, the example embodiment can be implemented for the dose set knob, that is, a dual screw dose set knob, to shorten an injection pen even more.

The technical solution to the technical problem of reducing drive train form factor for a plunger in a pen employs a screw drive mechanism and related principles such as lifting torque, efficiency, thread pitch, and friction, where lifting torque is a function of applied axial load (force or pressure), thread pitch, friction parameters, and diameter. In some example embodiments, the related mechanical equations may be further expanded to capture the full details of thread geometry such as flank and lead angle, and many other special parameters. ACME threads can be generally used to tweak the balance of lifting torque, power required, efficiency, and other functional parameters such as smoothness of operation and cost.

The example embodiments are further advantageous because they employ anti-rotation features for the screw-like components in the plunger rod drive assembly (e.g., the use of tongue and grooves for the double thread telescoping screw-train embodiment in FIGS. 6-10, and the use of a slotted anti-rotation sleeve for the double-acting telescoping screw-train embodiment in FIGS. 11-14).

Example embodiments of the disclosure may address at least the above problems and/or disadvantages and other disadvantages not described above. Also, example embodiments are not required to overcome the disadvantages described above, and may not overcome any of the problems described above.

Example embodiments of the present disclosure provide system components that can facilitate a reduction in the overall size or footprint of a drug delivery device, such as a medication injection pen, by a configuration of a container, cartridge, vial, reservoir or barrel for a medium or fluid with a mechanism or driving components for advancing a plunger to dispense the medium or fluid from the container, cartridge, vial, reservoir or barrel, where the mechanism or driving components can be disposed such that the overall length of the driving components can be reduced compared to conventional designs.

Matters of these exemplary embodiments that are obvious to those of ordinary skill in the technical field to which these exemplary embodiments pertain may not be described here in detail. In addition, various features of the exemplary embodiments can be implemented individually or in any combination or combinations, and would be understood by one of ordinary skill in the art of medicament delivery devices.

As would be readily appreciated by skilled artisans in the relevant art, while descriptive terms such as “fluid”, “medication”, “medium”, “stopper”, “plunger”, “thread”, “syringe”, “driver”, “screw”, “gear”, “piston”, “top”, “side”, “bottom,” “upper,” “lower,” “proximal”, “distal”, “container”, “reservoir”, “chamber” and others are used throughout this specification to facilitate understanding, it is not intended to limit any components that can be used in combinations or individually to implement various aspects of the embodiments of the present disclosure.

It will be understood by one skilled in the art that this disclosure is not limited in its application to the details of construction and the arrangement of components set forth in the above description or illustrated in the drawings. The embodiments herein are capable of other embodiments, and capable of being practiced or carried out in various ways. Also, it will be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings. Further, terms such as up, down, bottom, and top are relative, and are employed to aid illustration, but are not limiting.

The above-presented description and figures are intended by way of example only and are not intended to limit the illustrative embodiments in any way except as set forth in the following claims. It is particularly noted that persons skilled in the art can readily combine the various technical aspects of the various elements of the various illustrative embodiments that have been described above in numerous other ways, all of which are considered to be within the scope of the claims.

1. A medication injection pen, comprising
a chamber to hold fluid, the chamber comprising an outlet from which the fluid is expelled, and a stopper configured to advance within the chamber to push fluid from the chamber through the outlet to a patient; and
a housing connected to the chamber and comprising a plunger rod subassembly configured to controllably move axially to move the stopper a selected amount in the chamber to expel a selected dose of the fluid;
wherein the plunger rod subassembly comprises at least two nested plunger rod members having a first plunger rod member slidably mounted within a second plunger rod member, and being controllably operated to move the first plunger rod member relative to the second plunger rod member to axially extend the plunger rod subassembly to push the stopper via a distal end of one of the nested plunger rod members.

2. The medication injection pen according to claim 1, wherein the housing further comprises:
a dose set knob that rotates with respect to the housing during dose setting; and
a driver selectively engaged with the dose set knob and threadably engaged with the plunger rod subassembly, the driver rotatably fixed with respect to the housing during dose setting;
wherein the driver moves axially with the dose set knob during dose setting, rotates with the dose set knob during an injection to cause axial movement of at least one of the nested plunger rod members.

3. The medication injection pen according to claim 1, wherein the chamber is a cartridge of fluid comprising the stopper and a subchamber holding the fluid, and the length of the plunger rod subassembly is less than the length of the subchamber when in an initial nested position and at least as long as the length of the subchamber when extended from the nested position.

4. The medication injection pen according to claim 1, wherein the first plunger rod member and the second plunger rod member are a plunger rod and a plunger rod inner member, respectively, that are arranged to be at least partially concentric with respect to each other such that the plunger rod at least partially surrounds at least a portion of the plunger rod inner member, and the plunger rod and the plunger rod inner member are controllably operated to move telescopically with respect to each other.

5. The medication injection pen according to claim 4, wherein the housing comprises a lead screw driver operable to be controllably rotated when the dose of fluid is to be delivered, the plunger rod inner member comprises inner threads that engage with outer threads on the lead screw driver to rotate when the lead screw driver rotates, the plunger rod inner member comprises outer threads that engage with inner threads in the plunger rod, and
wherein the inner threads and outer threads of the plunger rod inner member, the inner threads in the plunger rod, and the outer threads on the lead screw driver have the same pitch.

6. The medication injection pen according to claim 4, wherein the plunger rod inner member comprises distally located outer threads that engage with inner threads in the plunger rod, the outer threads being one of male threads and female threads and the inner threads being the other one of male threads and female threads.

7. The medication injection pen according to claim 6, wherein the plunger rod is arranged within the housing to be prevented from rotating within the housing and relative to the plunger rod inner member, and the housing further comprises a lead screw driver operable to be controllably rotated when the dose of fluid is to be delivered, and
the plunger rod comprises a counterbore at its proximal end that prevents a distal end of the plunger rod inner member from disengaging from the plunger rod,
the plunger rod inner member comprises
a cap at its distal end,
inner threads that engage with outer threads on the lead screw driver to rotate when the lead screw driver rotates, and
a coupling mechanism configured to provide temporary engagement between the plunger rod inner member and the lead screw driver until rotation of the plunger rod inner member causes the plunger rod to move axially to a point where the cap abuts the counterbore of the plunger rod and creates a force to disengage the plunger rod inner member from the lead screw driver and allow the plunger rod inner member to move axially.

8. The medication injection pen according to claim 7, wherein the coupling mechanism on the plunger rod inner member comprises at least one detent on a proximal end thereof, the at least one detent configured to be frictionally engaged with a correspondingly-shaped and snap-fitted notch provided on a distal surface of the lead screw driver.

9. The medication injection pen according to claim 7, wherein the housing comprises a brake tower axially and rotatably fixed thereto, the brake tower comprising a plurality of slots, the plunger rod comprising at least one tongue extending into one of the slots to engage the plunger rod with the brake tower, the plunger rod inner member comprising at least one tongue on its proximal end that enters the nearest one of the slots after the plunger rod inner member disengages from the lead screw driver to stop further rotation of the plunger rod and the plunger rod inner member and allow axial advancement of the plunger rod inner member as the leadscrew rotates.

10. The medication injection pen according to claim 4, wherein the housing comprises a brake tower axially and rotatably fixed thereto, and a proximal end of the plunger rod inner member and the brake tower have same handed threads that engage each other, the same handed threads being one of clockwise threads and counterclockwise threads, and
a distal end of the plunger rod inner member and the plunger rod have same handed threads that engage each other and are the other one of clockwise threads and counterclockwise threads such that the plunger rod and the plunger rod inner member move at the same time when a lead screw driver engaged with the brake tower and the plunger rod inner member rotates.

11. The medication injection pen according to claim 10, wherein the threads on the plunger rod inner member, the plunger rod, and the brake tower have the same pitch.

12. The medication injection pen according to claim 10, wherein the housing further comprises an anti-rotation sleeve disposed between the brake tower and the plunger rod, the anti-rotation sleeve comprising a slot, and the brake tower and the plunger rod each comprising a stop member that is received in the slot.

13. The medication injection pen according to claim 10, wherein an axial cross-section of the lead screw driver comprises at least a portion thereof that is shaped to be keyed into an internal portion of the plunger rod inner member.
